(19) United States
(12) Patent Application Publication
(10) Pub. No.: US 2026/0305952 A1
(43) Pub. Date: Oct. 8, 2026

(54) MONITORING SYSTEM AND METHOD FOR MEASURING A QUANTITY OF BLOOD LOST AFTER VAGINAL DELIVERY

(71) Applicants: INSTITUT NATIONAL DE LA SANTE ET DE LA RECHERCHE MEDICALE, Paris (FR); UNIVERSITE PARIS CITE, Paris (FR); FONDATION HOPITAL SAINT-JOSEPH, Paris (FR)

(72) Inventor: Elie Azria, Paris (FR)

(73) Assignees: INSTITUT NATIONAL DE LA SANTE ET DE LA RECHERCHE MEDICALE, Paris (FR); UNIVERSITE PARIS CITE, Paris (FR); FONDATION HOPITAL SAINT-JOSEPH, Paris (FR)

(21) Appl. No.: 19/474,903

(22) PCT Filed: Apr. 12, 2024
(86) PCT No.: PCT/EP2024/059971

§ 371 Date: Oct. 13, 2025

(30) Foreign Application Priority Data
Apr. 14, 2023 (EP) 23305574.8

(51) Int. Cl. A61B 5/02; A61B 5/00; G01F 22/00; G01G 3/14; G01G 21/23

(52) U.S. Cl. CPC A61B 5/02042; A61B 5/4343; A61B 5/7278; A61B 5/742; G01F 22/00; G01G 3/14; G01G 21/23

(57) ABSTRACT

This monitoring system (4), for measuring a quantity of blood lost by a woman having given birth vaginally, includes a port-partum blood flow collection container (42), a weighing device (44, 446) configured to automatically and continuously weigh blood present in the container, a computing unit (82) configured to automatically compute a blood volume (Vb) in the container (42) and a blood flow rate (Fb) of blood flowing into the container, on the basis of an output signal (S446) of the weighing device. The monitoring system (4) also includes a screen (48) configured to display both a first value of the blood volume (Vb) in the container (42), computed by the computing unit (82), and a second value of the blood flow rate (Fb), also computed by the computing unit (82).

### TECHNICAL FIELD OF THE INVENTION

The present invention concerns a monitoring system and a monitoring method for measuring a quantity of blood lost by a woman having given birth vaginally, which allows detecting a risk of post-partum haemorrhage.

### BACKGROUND OF THE INVENTION

One danger of childbirth is the risk of a significant loss of blood by the mother, after the actual delivery, i.e. post-partum. This phenomenon is known as post-partum haemorrhage or PPH. Post-partum haemorrhage meets the definition of blood loss within 24 hours of birth of more than 500 mL. The incidence of PPH is between 5 and 10% of births. Worldwide, PPH is the leading cause of maternal mortality, severe acute maternal morbidity, and maternal admission to intensive care. Early diagnosis and rapid intervention are considered key elements in reducing the risk of progression to severe PPH, defined as blood loss larger than 1000 mL, and maternal mortality. Currently, nearly all maternal deaths from hemorrhage are considered preventable.

It is known that blood loss estimated visually without any tool is greatly underestimated and leads to delays in diagnosis and management. In order to have an objective assessment of blood loss, the use of graduated collection bags placed under the women's wombs after delivery has become widespread in high income countries. The use of these bags is not systematically recommended for post-partum monitoring, but is recommended by several professional societies in case of hemorrhage to estimate blood loss.

Such collection bags might be graduated and partly or fully transparent, in order for a practitioner to estimate the blood volume present in a bag by looking at the bag and its scaling. This practitioner may be a nurse, a midwife or an obstetrician gynecologist. Depending on the blood volume in the collection bag, the practitioner is supposed to identify a suspicion of post-partum haemorrhage.

This prior art system is not reliable in so far as the estimate by the practitioner might be biased by a reading error on the scaling of the collection bag, or by rubbish present in the collection bag, which leads the practitioner to overestimate or underestimate the quantity of blood present in the collection bag.

Moreover, the practitioner may be disturbed by external factors, such as another emergency, and might be prevented from visually checking the collection bag and its content for a relatively long period.

Another issue with the prior art system is that the practitioner has to stop the patient's care for checking the content of the collection bag.

Furthermore, with the prior art system, the practitioner estimates only the quantity of blood in the collection bag.

Moreover, the system of the prior art depends on the practitioner in charge of monitoring the parturient woman, which is not optimal, in terms of reproducibility and safety, especially when the practitioner works for a long period, e.g. during a night shift.

US2020/0155016A discloses an apparatus and a method for detecting and notifying postpartum haemorrhage where a closed bag rests on a weighing scale. The pipe between the patient and the closed bag may become clogged. The flowrate of blood into the bag is limited by the fact that it is closed. Soaked pads cannot be disposed in this bag and the blood they contain is therefore not taken into account when estimating blood loss. Moreover, the delivery table must be modified in order for the pipe to go from the patient to the closed bag. The scale and the closed bag may be a limitation in terms of mobility in the event of an emergency transfer to the operating theatre or interventional radiology room.

On the other hand, CN11040357A discloses a blood collection tray configured to be positioned under a patient's buttocks and equipped with means for measuring the weight of blood collected. This tray may be uncomfortable for the patient and the maximum volume of collected blood is relatively low.

With the known devices, a practitioner has no precise information on the blood flow rate, whereas this flow rate can indeed be also representative of a potential haemorrhage. Actually, a high blood flowrate might be a sign of a haemorrhage, even if the quantity of blood already collected in the collection bag is still below 500 mL.

### SUMMARY OF THE INVENTION

The present invention aims at solving these issues with a better detection of a risk of post-partum haemorrhage, which avoids relying on the concentration and availability of the staff in charge of a parturient woman and which allows detecting a potential haemorrhage even if the quantity of blood in a collection container is low.

To this end, the invention relates to a monitoring system for measuring a quantity of blood lost by a woman having given birth vaginally. This monitoring system includes a port-partum blood flow collection container and a weighing device configured to automatically and continuously weigh blood present in the container. A computing unit is configured to automatically compute a blood volume in the container, on the basis of an output signal of the weighing device. A screen is configured to display a least one of a first value of the blood volume in the container, computed by the computing unit. The computing unit is also configured to automatically compute a blood flow rate of blood flowing into the container. The screen is configured to display a second value of the blood flow rate, also computed by the computing unit.

Thanks to the invention it is possible to know precisely, in real time, a quantity and a flowrate of blood lost by a new mother during a given period of time, after delivery, in an objective way, without over-or under-estimation by a practitioner and even if a practitioner has to work with another patient at the same time. The invention is based on the assumption that weighing of the blood collected post-partum allows precisely knowing the quantity of blood lost by the new mother and that a blood flow rate, computed on the basis of variations of the collected blood weight, is likely to be representative of a suspicion of haemorrhage situation if this flow rate is high. This allows the practitioner to react in case of high blood flow rate, before the quantity of blood lost by the patient reaches a threshold value.

According to advantageous but non-compulsory aspects of the invention, such a monitoring system might incorporate one or several of the following features:
The weighing device includes a frame for supporting the container and a weight sensor mounted between two parts of the frame.
The frame includes a first part provided with immobilization means for immobilizing the first part on a delivery table and a second part provided with holding means for holding the container and the weight sensor includes at least one strain gauge connecting the first part and the second part of the frame.
The container is a flexible bag and the holding means include at least one clip configured to clip an edge of the flexible bag in a configuration where the flexible bag hangs down from the frame, without touching on the ground.
The computing unit belongs to an electronic unit configured to handle an output signal of the weight sensor and to provide the screen with a signal representative of the instantaneous blood volume in the container and of the instantaneous blood flow rate toward the container.
The electronic unit includes an amplification module for amplifying the output signal of the weight sensor and a communication module for sending to the screen a signal including a data set with the blood volume and the blood flow rate computed by the computing unit.
The electronic unit is housed in a waterproof and cleanable according to hospital standards casing.
The weighing device is incorporated in a scale, which supports the container.

According to a second aspect, the invention relates to a monitoring method for measuring a quantity of blood lost by a woman having given birth vaginally, this method including collection of post-partum blood flow in a container, wherein this method includes at least the following steps:
a) automatically and continuously weighing a quantity of blood present in the container; and
b) automatically computing a volume of the blood present in the container, based on a weight determined at step a);
c) automatically computing a flow rate of the blood flowing into the container, on the basis of variations of the volume computed at step b); and
d) displaying both
   a first value of the volume computed at step b),
   a second value of the blood flow rate computed at step c). as well as two curves showing the temporal trend in blood volume and flow rate.

The method of the invention allows keeping a practitioner aware of the situation of a parturient woman with respect to a potential post-partum haemorrhage in an objective and continuous way. In particular, the practitioner's attention can be drawn to a risky situation by a configurable alarm. This method provides the practitioner with reliable information, so that the practitioner can determine if post-partum haemorrhage is taking place, based on the displayed information, on his/her knowledge of the parturient woman, her health record and physical condition.

According to further advantageous aspects of the invention, the method of the invention might incorporate one or several of the following features:
The blood volume is computed at step b) on the basis of a measure of the weight of the container.
The method includes at least the following steps consisting in:
   e) comparing a value of the blood volume to a first threshold value and/or a value of the blood flow rate to a second threshold value;
   f) adapting step d) according to the result of the comparison of step e).
An alarm is displayed at step d) if the result of the comparison of step e) shows that the blood volume value is above the first threshold value and/or the blood flow rate value is above the second threshold value.
The method includes a step of smoothing over time instantaneous values of the weight of the container, of the blood volume and/or of the blood flow rate.
The method includes at least the following steps consisting in:
   g) taking into account the fact that an object has been thrown in the container;
   h) subtracting the weight of the object thrown in the container from the weight determined at step a).
Step d) occurs on a screen and the method includes a step i), prior to step d), consisting in transmitting to the screen a signal including data to be displayed and relating to the blood volume computed at step b) and to the blood flow rate computed at step c), whereas the signal is preferably transmitted wirelessly at step i).
Step d) includes showing a curve representative of the blood volume computed at step b) and/or a curve representative of the blood flow rate computed at step c).

### BRIEF DESCRIPTION OF THE DRAWINGS

The invention will be better understood, based on the following description, which is given in correspondence with the appended figures and as an illustrative example, without restricting the object of the invention. In the annexed figures:

FIG. 1 is a schematic representation of a delivery room including a monitoring system according to the invention;

FIG. 2 is a schematic representation of the monitoring system used in the delivery room of FIG. 1;

FIG. 3 is a perspective view, from an angle different from the one of FIG. 2 and on larger scale, of a frame of the monitoring system of FIG. 2;

FIG. 4 is an electronic diagram showing some components of the monitoring system of FIGS. 2 and 3;

FIG. 5 is a block diagram of a monitoring method implemented with the monitoring system of FIGS. 2 to 4; and

FIG. 6 represents a screen of the monitoring system of FIGS. 2 to 4 in three different situations.

### DETAILED DESCRIPTION OF SOME EMBODIMENTS

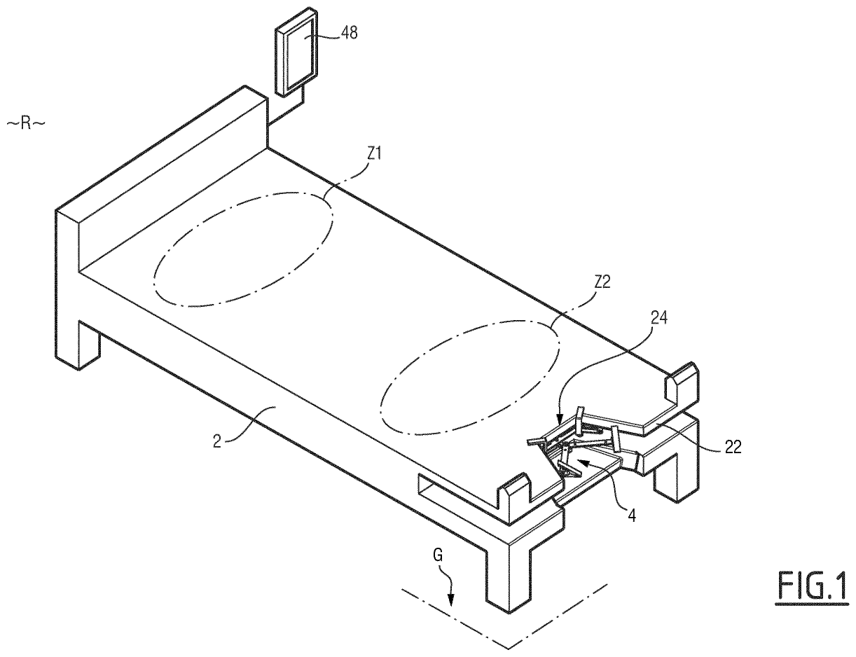

FIG. 1 shows a delivery room R of a hospital, where a delivery table 2 rests on the ground G.

The delivery table defines a first zone Z1 for accommodating the head of a parturient woman and a second zone Z2 for accommodating the pelvis of the parturient woman giving birth vaginally.

According to the invention, the delivery table 4 is equipped with a monitoring system 4, which allows automatically measuring a quantity of blood lost by the parturient woman.

This monitoring system includes a blood collection container 42, a frame 44, an electronic unit 46 and a screen 48 attached to the delivery table 2.

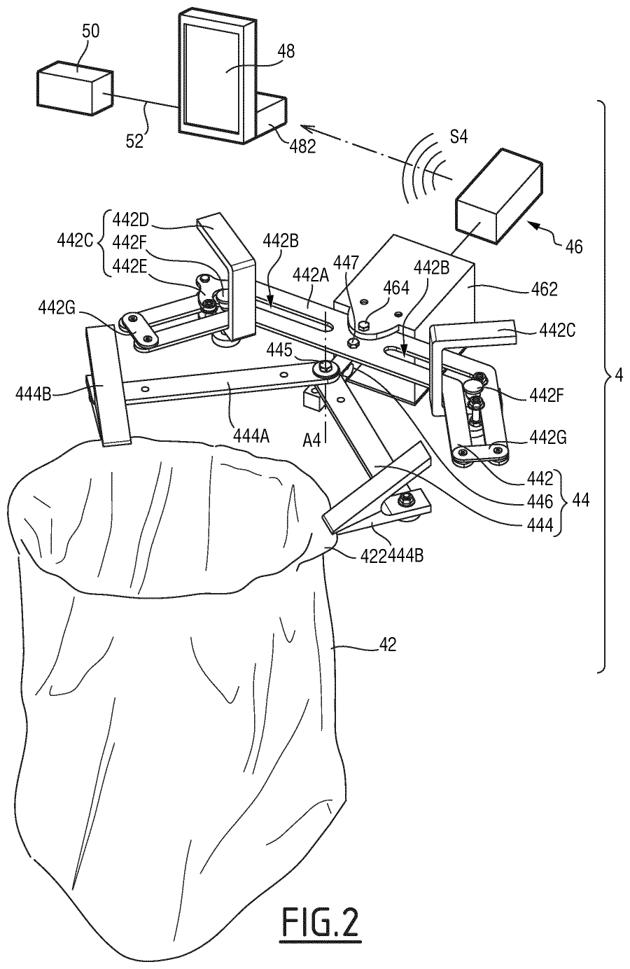

For the sake of simplicity, the container 42 is represented on FIG. 2 only. In the example of the figures, this container is a flexible plastic bag. This bag 42 is liquid proof and strong enough to resist a weight of several kilograms (kg), e.g. 5 or 10 kg.

The bag 42 does not need to be transparent nor to be provided with a scaling or graduation.

Hereafter, the container 42 is sometimes referred to as “the bag 42”.

The frame 44 is configured to be mounted on an edge 22 of the delivery table 2, at the level of a notch 24 formed on the delivery table, next to the zone Z2.

The frame 44 includes a first part 442, a second part 444 and a weight sensor 446, which forms a connection rod between the first part 442 and the second part 444.

The first part 442 includes a rigid metallic plate 442A made in a flat sheet of metal, for instance stainless steel, and provided with two slots 442B where two clamps 442C can respectively slide, in order to be adjusted along the edge 22, at the level of the notch 24.

Generally speaking, the shape of the plate 442A follows the shape of the edge 22 at the level of the notch 24.

Each clamp 442C includes a globally U shaped bracket 442D and a slider 442E which carries a pressure screw 442F. For the sake of simplicity, the slider of one clamp 442C is not represented on the right of FIG. 2.

Two end lockers 442G are provided at the opposite ends of the plate 442A in order to prevent the clamps 442C from falling out of the slots 442B. The end lockers are optional.

The structure of the first part 442 allows immobilizing the frame on the edge 22, at the level of the notch 24, by clamping this edge between the brackets 442D and the pressure screws 442F of the two clamps 442C.

The second part 444 includes two arms 444A articulated on a common articulation axis A4 defined at one end of the weight sensor 446.

Each arm 444A carries holding means for attaching the bag 42, in the example a clip 444B capable of clipping an edge 422 of the bag 42.

Advantageously, the clips are non-contending clips.

The possibility of articulation of the two arms 444A around the axis A4 allows adapting the positions of the two clips 444B to the geometry of the bag 42 and to the width of the notch 24.

Advantageously, the second part 444 is mounted on the weight sensor 446 by a first screw 445, which defines the articulation axis A444. On the other hand, the weight sensor 446 is mounted on the first part 442 by a second screw 447.

Advantageously, the weight sensor 446 forms a connection rod and holds, in a cantilever way, the second part 444 with respect to the first part 442 when this first part is immobilized on the delivery table 2 via the clamps 442C.

The size of the container-forming bag 42 and the location of the frame 44 on the delivery table 2 are chosen so that, when the bag is hung on the frame 44 by the clips 444B, it does not touch the ground G.

The weight sensor 446 is formed of four combined strain gauges 446A, 446B, 446C and 446D. For example, the weight sensor can be a weight sensor marketed by Sparkfun under reference TAL22B. This sensor covers the range of weight expected for the blood in the container, between 0 and 3 kg.

Due to its location between the first and second parts 442 and 444 of the frame 44, the weight sensor 446 can continuously determine the combined weight of the second part 444, plus the bag 42 and its content.

The electronic unit 46 is housed in a waterproof casing 462 mounted on the first part 442 via a screw 464. This casing 462 protects the electronic unit 46 from liquid or vapor projections during delivery or upon cleaning of the delivery table 2. This casing 462 is cleanable according to hospital standards.

On FIG. 2, the electronic unit 46 is represented outside the casing 462, in order to better show this unit. In practice, the electronic unit 46 is located within the waterproof casing 462 when the monitoring system 4 is in use.

The electronic unit 46 includes a battery set 80, which feeds a computing unit 82 with electric power, with a continuous voltage, e.g. 5 volts (V). For instance, the battery set 80 includes two lithium batteries, in series, sized to provide the electronic unit 46 with an autonomy of several days.

A microprocessor 822 belongs to the computing unit 82 and is configured to execute a computer program stored in a memory 824 of the computing unit 82.

The battery set 80 also supplies an amplification module 84 with electric power. This amplification module 84 magnifies the small resistance variations corresponding to deformations of one or several of the strain gauges 446A-446D, so that these deformations can be detected by the computing unit 82. The amplification module 84 is, for example, a communication module sold by Avia Semiconductor under reference HX711. The amplification module 84 communicates with the computing unit 82 via two digital lines L84 and L′84.

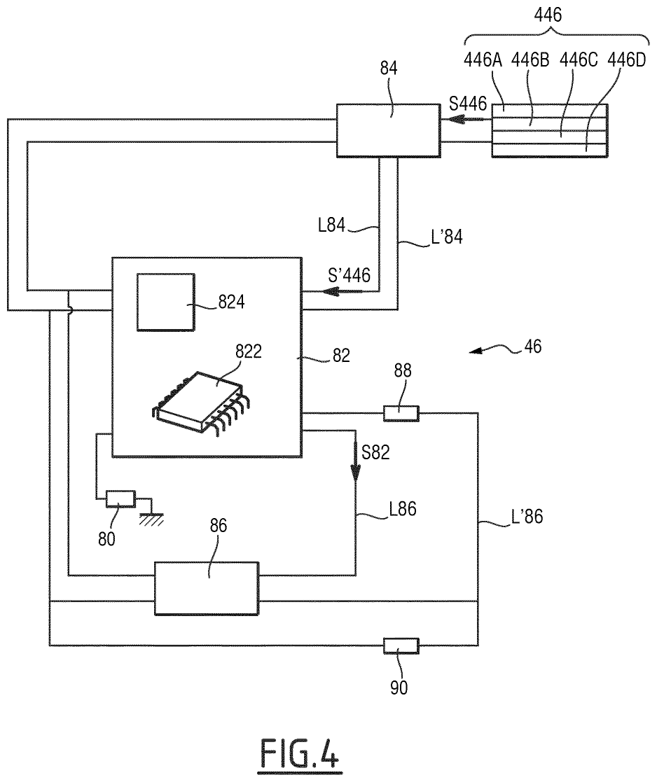

Preferably, as shown on FIG. 4, the amplification module 84 is supplied with electric power from the battery set 80, through the computing unit 82. The electronic unit 46 also includes a transmission module 86 provided with electrical power from the battery set, preferably through the computing unit 82. The transmission module 86 is, for example, a bi-directional Bluetooth transmission module sold by the company Hackspark under reference HC05. The transmission module 8 communicates with the computing unit by two digital lines L86 and L′86. Digital line L86 corresponds to the transmission pin of the communication module 86, whereas digital line L′86 corresponds to the reception pin.

Two resistors 88 and 90 are also provided in the electronic unit 46, respectively on digital line L′86 and on a voltage return line, in order to lower the voltage supplied to the communication module, e.g. from 5 V to 3.3 V, according to its working specification.

The monitoring system 4 allows the weight sensor 446 to continuously and automatically monitor the weight of the second part 444 equipped with the container 42. This information is included in an output signal S446 of the weight sensor 446, which is amplified by the amplification module 84. This information is conveyed, as a part of the amplified output signal S′446 of the weight sensor 446 and in the digital line L84, to the computing unit 82 where it is processed by the microprocessor 822.

The combined weight of the second part 444 and the container 42 is known from the manufacturing process of the monitoring system 4 and from the container specification. Thus, the weight sensor 446 can continuously and automatically determine the net weight of the content of the container 42, which is considered to be formed mainly of blood.

Knowing the density of blood, the microprocessor 822 automatically computes, in real time, the instantaneous volume Vb of blood present in the bag 42 from the net weight of the content of the container.

The collection of the combined weight of the second part 444, the container 42 and its content, that is the treatment of the output signal S446 in order to compute the blood volume Vb, is preferably performed on a regular basis, at a given frequency, compatible with the working speed of the microprocessor 822.

The output signal S446 of the weight sensor 446 is also used by the computing unit 82 to automatically compute, in real time, an instantaneous blood flow rate Fb flowing into the container 42. This blood flow rate Fb corresponds to the variations of the blood volume Vb over time and can be computed by the microprocessor 822 at a given frequency, preferably the same frequency as the computing frequency of the blood volume Vb.

The blood volume is a cumulative value, which corresponds to the blood accumulated within the container 42. Thus, this volume value increases over time. On the contrary, the flow rate value can increase or decrease over time.

The results of the automatic computation process performed by the microprocessor 822, in particular the values of the instantaneous blood volume Vb and of the instantaneous blood flow rate Fb, are included, as a data set, in an output signal S82 of the computing unit 82 and conveyed via the digital line L86 to the communication module 86. This data set is included in a signal S4 sent by the electronic unit 46 to the screen 48, via the communication module 86.

The screen 48 is equipped with a Bluetooth communication module 482 adapted to receive and handle the signal S4 emitted by the communication module 86.

In any case, irrespective of the type of the communication module 86, the screen 48 is equipped with a communication module compatible with the communication module 86 of the electronic unit 46.

The screen 48 is configured to process the data set included in the signal S4 and to automatically and continuously show and information extracted from this data set, in real time. As explained here above, this information is representative of the instantaneous blood volume Vb in the container formed by the container-forming bag 42 and of the instantaneous blood flow rate Fb arriving in this bag.

Therefore, the monitoring system 4 of the invention allows automatically and continuously displaying an information relating to the blood volume present in the container 42, which corresponds to the quantity of blood lost by the parturient woman. This system also allows automatically and continuously displaying the flow rate of the blood losses, which can be critical in some circumstances, as mentioned here above.

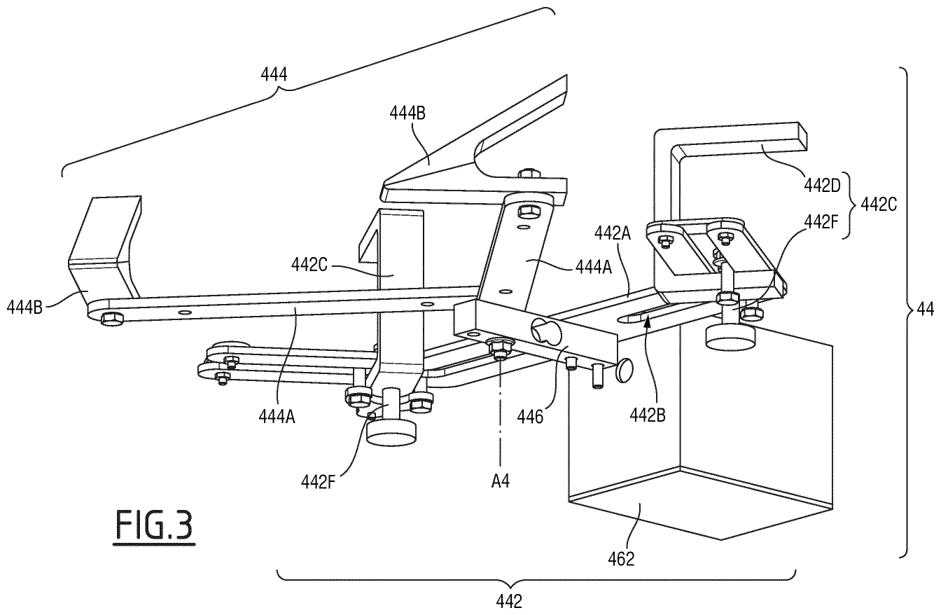

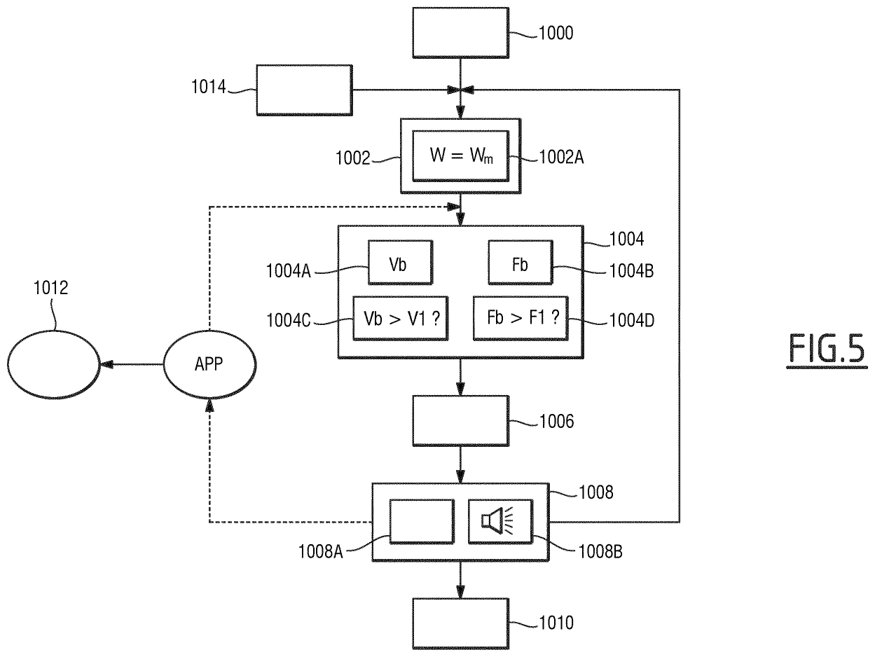

FIG. 5 shows a method for measuring a quantity of blood lost by a woman having given birth vaginally, which is according to the invention and implemented with the monitoring system 4 of FIGS. 2 to 4.

This method includes a first initialization step 1000, launched by a practitioner, which corresponds to the initialization of the monitoring system 4. In step 1000, the communication between the computing unit 82 and the communication module 86 is initiated through lines L86 and L′86 and the communication between modules 86 and 482 is also initiated. In addition, the container 42 is hung to the second part 444 of the frame 44, without touching the ground G, and the weight sensor 446 is calibrated, with the net weight value included in the signal S446 set to zero.

In a second step 1002, a variable “weight” or W is acquired by the microprocessor 822, from the output signal S446 of the weight sensor 446.

Preferably, in an elementary step 1002A of the step 1002, this variable W is assigned to an average Wm of several net weight values detected via the weight sensor 446 over time. For example, the microprocessor 822 of the computing unit 82 can be programmed to collect a net weight value through the weight sensor 446 every 10 milliseconds (ms), that is with a frequency of 100 Hz, and to average the collected values over 100 ms. This smoothing or averaging of the weight values W, performed at elementary step 1002A, allows smoothing of the blood volume and blood flow rate values, Vb and Fb, as explained here below.

The interval between the collection of two weight values and the duration used for averaging, in other words smoothing, these values can be different respectively from 10 and 100 ms, and depend on the programming of the microprocessor 822.

In a third step 1004 of the method, the blood volume Vb present in the container 42 is computed, together with the value of the blood flow rate Fb. Computation of the volume value Vb takes place in an elementary step 1004A of step 1004, on the basis of the variable W assigned at step 1002. Computation of the blood flow Fb rate takes place in another elementary step 1004B of step 1004, where the computing unit 82 establishes the derivative of the function representing the value of the blood volume over time.

Since the value W has advantageously been smoothed at step 1002A, the values Vb and FB are also smoothed. Smoothing of values W and Vb allows buffering, that is absorbing, sharp variations due to interactions between the bag 42 and the practitioner or to movements of the parturient woman during delivery.

According to a variant of the invention, no smoothing of the weight variable W occurs at step 1002 but smoothing occurs at step 1004, after elementary steps 1004A and 1004B.

Smoothing of the weight values W and/or of the blood volume and flow rate values Vb and Fb is advantageous for at least two reasons. First, it avoids that the values displayed on the screen 48 change many times and very quickly over time, which could make reading of these values difficult for a practitioner. Second, it allows taking into account an external phenomenon, such as when the practitioner hits the frame 44 during examination of the parturient woman, which results in a transitory acceleration sensed by the strain gauges of the weight detector 446. This acceleration temporarily disturbs the weight value W detected by the weight sensor 446 and induces non-representative variations of the values Vb and Fb. Smoothing of the values W, Vb and/or Fb allows masking these non-representative variations and avoids alerting the practitioner for undue reasons.

However, according to another variant of the invention, no smoothing of the values W, Vb and Fb takes place, since smoothing is optional.

Once the blood volume and the flow rate have been computed in step 1004, they are transferred to the communication module 86 in the output signal S82 and sent to the screen 48, as part of the signal S4, by wireless communication between the communication modules 86 and 482. This corresponds to a fourth step 1006 of the method.

Once received by the screen 48, the values of the blood volume and blood flow rate are treated and displayed on the screen 48, in a fifth step 1008 of the method.

Then, the system comes back to step 1002 as shown by the return arrow on the right of FIG. 5. In other words, the method works by iteration between steps 1002 and 1008, which allows continuously displaying updated values of the blood volume and flow rate, Vb and Fb. These iterative steps 1002 to 1008 are implemented after delivery, possibly as soon as possible after delivery.

According to an optional aspect of the invention implemented in a sixth step 1010 of the method, the values of the blood volume Vb and the blood flow rate Fb displayed on the screen 48 are stored in a storing device, such as a hard disk 50 connected to the screen 48 by a wired link 52. This allows keeping a track of the data relating to post-partum blood losses for every patient.

Alternatively, the storage performed at step 1010 occurs in the memory of 824 of the computing unit 82 or in another storage space, which belongs to the electronic unit 46.

According to another optional aspect of the invention, the electronic unit 46 can communicate with an application APP be loaded on a smartphone or a tablet. This allows keeping a practitioner aware of the result of the monitoring method of the invention, even if this practitioner is not located in the delivery room R, as long as the practitioner keeps his/her smartphone or tablet with him/her. If such an application APP is used, storing of the same data as in step 1010 can occur in a further step 1012, via wireless connection between the application APP and a storage space, e.g. in the cloud.

Advantageously, artificial intelligence, for instance a neural network, is used for analyzing the data stored at step 1010 and/or 1012, in particular the flow rate data. A database may be used to train the artificial intelligence, based on the data collected, including the volume and flow rate of the blood losses, the shock index or other vital signs such as blood pressure and cardiac heart rate, with the purpose of predicting the evolution of the blood losses volume and flow rate curves over time, on the basis of the first values measured by the system of the invention.

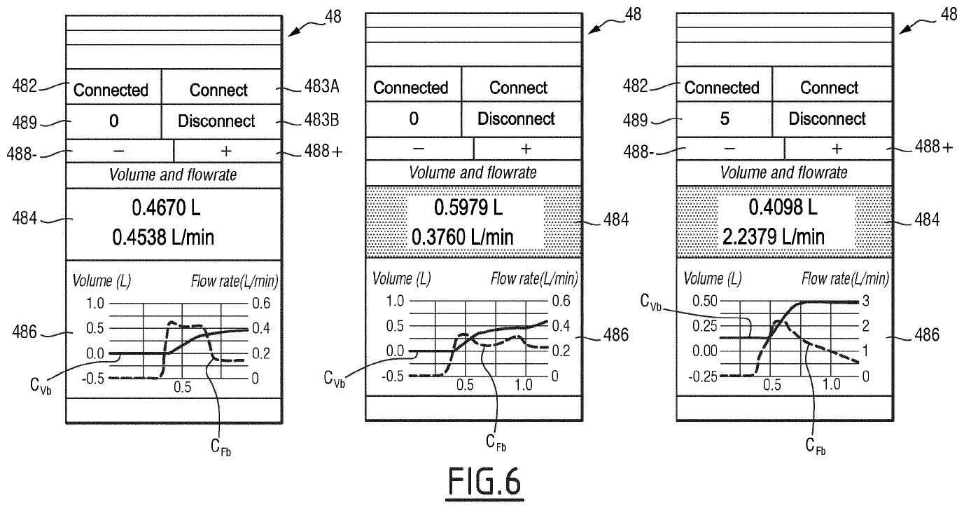

FIG. 6 shows an example of the screen 48 at step 1008. If the application APP is used, the screen of the smartphone or tablet of the practitioner might also look like FIG. 6.

The left part of FIG. 6 shows the screen 48 in case of a normal situation.

The method of the invention includes a step, non-represented on FIG. 5, where the screen 48 checks if it is actually connected to the electronic unit 46, as the result of the step 1000 mentioned here above. If such is the case, the word “connected” is displayed in a first display zone 482 of the screen 48, as shown on FIG. 6. If not, the practitioner is invited to connect the screen to the electronic unit be pressing on a tactile zone 483A of the screen.

The screen is advantageously a touch screen. In such a case, it includes two tactile zones 483A and 483B allowing to connect the screen to, or disconnect the screen from, the electronic unit 46

The screen 48 includes a second display zone 484 where the values of the blood volume Vb and blood flow rate Fb computed at step 1004 are displayed, as mentioned here above in connection with step 1008. In the situation represented on the left of FIG. 6, the blood volume detected in the container 42 is 0.4670 L and the blood flowrate at 0.5 second is 0.4538 L/min.

The screen 48 includes a third display zone 486 where a graphic representation of the blood volume Vb in the container 42 is displayed, as a curve C_{Vb} showing the value of the blood volume as a function of time. This allows the practitioner to get information very quickly by looking at the shape of the curve C_{Vb}, which might be very different depending on the circumstances, as can be derived from the comparison of the three zones 486 visible on FIG. 6.

The screen is also advantageously configured to display, in the display zone 486, a graphic representation of the blood flow rate Fb, in the form of a second curve C_{Fb} showing the value of the blood flow rate as a function of time.

As can be deduced by the comparison of the three situations represented on FIG. 6, the scales used in the third display zone 486, respectively on the left and on the right of the curves, can be automatically adapted to the values of blood volume Vb and blood flow rate Fb to be displayed. This explains why these scales are different between the first two situation and the third situation.

In practice, in order to provide more practical information to the practitioner, step 1004 advantageously incorporates at least one comparison step.

In an elementary step 1004C, the value of the blood volume Vb computed at step 1004A is compared to a first threshold value V1, for instance equal to 500 mL. The result of the comparison of step 1004C is included in the data set of the output signal S82 and transferred to the screen 48. The practitioner can consider that if less than 500 mL have been collected in the container 42, the risk for post-partum haemorrhage is relatively low and that the situation is normal. On the other hand, if the value Vb is larger than or equal to 500 mL, the practitioner may consider that there is a risk of post-partum haemorrhage.

If the value Vb is strictly larger than the first threshold value V1, the attention of the practitioner is drawn to this particular situation, in an elementary step 1008A of step 1008, by changing the color of the display zone 486. For instance, the background of the second display zone 484 can be turned to red at elementary step 1008A. This corresponds to the second situation represented in the center of FIG. 6 where the display zone 484 is shaded to represent to color change of the elementary step 1008A.

Similarly, the value of the flow rate Fb computed at step 1004B can be compared in an elementary step 1004D of step 1004, to a second threshold value F1 which can be set, for instance, to 0.5 L/min. The result of the comparison of step 1004D is also included in the data set of the output signal S82 and transferred to the screen 48.

If, during the comparison step 1004D, the flow rate is determined to be strictly larger than to this threshold value F1, then the display zone 484 changes color in elementary step 1008A. This corresponds to the third situation represented in the right of FIG. 6, where the display zone 484 is also shaded.

In other words, step 1008 is adapted at elementary step 1008A, in order to take into account the results of the comparisons of the elementary steps 1004C and 1004D. In practice, the elementary step 1008A corresponds to the triggering of a visible alarm for a practitioner looking at the screen 48.

Alternatively or cumulatively, an audible alarm may be triggered, at an elementary step 1008B of step 1008, if one of the comparisons of step 1004C and 1004D shows that the blood volume Vb or the flow rate Fb is strictly larger than the corresponding threshold value V1 or F1. This approach has the advantage of alerting the practitioner even if he/she is not looking at the screen.

According to an optional aspect of the invention, it is possible to take into account the fact that the practitioner might have to throw soaked pads or compresses in the container, after use. Here after “pad,” is used to designate a pad, a compresses, a gauze and any other object loaded with blood thrown into the bag 42. In such a case, the weight of such pads should not alter the accuracy of the weight determination of step 1002.

To this end, a step 1014 is advantageously provided in the method of the invention where the practitioner indicates when he or she throws a pad in the bag 42. This can be made with two tactile zones 488+ and 488− of the screen 48. By pressing one or several times on zone 488+, the practitioner can indicate how many pads have been thrown in the bag 42. The number of pads taken into account is shown in a fourth display zone 489 of the screen 48. If the practitioner realizes that the number of pads is not correct, he can decrease or increase this number by pressing on one of the tactile zones 488− or 488+ of the screen.

This approach and the possibility of connecting/disconnecting via zones 483A and 483B rely on the use of a touch screen, which is widely available nowadays and which is standard on smartphones. However, if necessary, a keyboard or another data entry device might be connected to the screen 48, if this screen is not a touch screen. For instance, a microphone can be used in order for the practitioner to use a voice command to indicate the number of soaked pads thrown into the bag 42.

The representation on the right of FIG. 6 shows a case where five pads have been thrown into the container 42.

Once a number of pads is known as a result of step 1014, their cumulative weight can be subtracted from the weight detected by the weight sensor 446, thus allowing to know the net weight of the blood present in the container and not to underestimate the volume of blood contained in the pads, at the end of step 1004A.

This approach relies on the fact that chirurgical pads have a standard weight, which is known and can be programmed in the computing unit 82, and stored in the memory 824.

The method of the invention is implemented automatically, once the initialization step 1000 has been launched. Thus, the instantaneous values of the blood volume Vb and blood flow rate Fb are continuously displayed on the screen 48, or on the screen of the smartphone or tablet when the application APP is used, and available for any practitioner in charge of the parturient woman.

The final diagnosis of post-partum haemorrhage is made by the practitioner, based on the information displayed on the screen 48 or on the screen of the smartphone/tablet. In other words, the method of the invention does not make a diagnosis but provides information to a practitioner who can make the diagnosis, taking into account external factors, such as the health condition and weight of the parturient woman.

In the meaning of the invention, the screen can a fixed screen, as the one represented with reference 48 on FIGS. 1 and 2 or the screen of a smartphone or tablet which can be kept by the practitioner with him or her, also when he is not physically present in the delivery room.

The invention is not limited to the example represented on the figures.

According to non-represented variants of the invention, the container 42 can be different from a flexible plastic bag.

The structure and geometry of the support frame 44 might be different from the one shown on the figures and discussed here above. In particular, the clips 444B can be replaced by other holding means. Alternatively, the second part 444 is made by a closed sub-frame, for instance with a regular shape, similar to the ones used for holding bags of waste in a dustbin. Alternatively, the second part of the frame may be formed of a curved semi-cylindrical sheet of metal. The clips 444B might also be replaced by hooks or similar holding devices.

In a variant, the number of strain gauges of the weight sensor 446 can be different from four, e.g. equal to one or two. For instance, the second part 444 of the frame can be directly mounted on the first part 442 and a strain gauge, preferably of the same kind as the strain gauge 446, can be mounted between each arm 444A and the clip 444B supported by this arm. In such a case, the weight sensor 446 includes two strain gauges.

According to another variant of the invention, another type of weight sensor can be used, e.g. a single point load cell, a S-beam load cell or a bending beam load cell.

According to another variant of the invention, the communication between the electronic unit 46 and the screen 48 is made by Wi-Fi or a wire communication is installed between the electronic unit 46 and the screen 48. Communication modules 86 and 482 are then adapted.

The computing unit 82 is represented on FIG. 2 as a single entity, such as a printed circuit board. Alternatively, it can be made of several physical entities connected together or of a module of a computing algorithm.

The weighing device of the invention should be capable of weighing a mass between 0 and 3 kg with a relatively good precision, in the order of 1 to 10 g.

The threshold values V1 and F1 are given purely as examples. They can be chosen by the practitioners in charge of the parturient woman, depending on their working habits and/or on the health record of the parturient woman.

According to another variant of the invention, the step 1004 includes the computation of only one value, among the blood volume Vb and the blood flow rate Fb. In such a case, only the value computed at step 1004 is displayed at step 1008.

According to a non-represented variant of the invention, the screen 48 shows the state of charge of the battery set 80 or an alarm is triggered when this state of charge is below a preset value.

According to another non-represented variant of the invention, the battery det 80 is replaced by a wire connection to the mains.

Alternatively, instead of using the tactile zones 488+ and 488−, the practitioner can indicate orally to the system how many pads have been thrown in the bag 42. This implies that a microphone is included is the system, for instance integrated to the screen 48 or part of a smartphone, that and a voice message handling program is used.

In any case, the number of soaked pads thrown into the bag 42 is known and taken into account in real time to compute the blood volume Vb and the blood flow rate Fb in the computing unit 82.

In a non-represented variant of the invention, the screen 48 is not attached to the delivery table 2 but supported by an independent post. According to another non-represented variant of the invention, the whole system 4 is independent from the delivery table 2. This makes the system of the invention independent from the type of the delivery table.

According to a non-represented aspect, the method of the invention includes a preliminary step where the weight sensor is calibrated. This can be done via a specific computer program and allows, in particular, taking into account ambient temperature variations. Such a calibration can be made before the first use of the system and, preferably, on a regular basis.

According to the invention, the weighing device formed by the frame 44 or the scale located below the container, including its weight sensor 446, is configured to automatically and continuously weigh the blood present in the container 42. The computing unit 82 automatically and continuously determines, in real time, the volume Vb and flow rate Fb of the blood losses and provides data to the screen 48 to keep a practitioner aware of the two essential parameters for post-partum haemorrhage. These parameters can be continuously displayed, in real time, on the screen, which allows the practitioner accessing the screen to be aware of these parameters at any time, to make an intellectual diagnosis on the basis of precise data and to react quickly in case of a post-partum haemorrhage.

The system 4 of the invention can be installed on a delivery table 2 instead of a known system supporting a graduated collection bag, without substantial modification to the delivery table. Existing delivery tables can easily be retrofitted with the system of the invention, without the need of drilling a hole for a connection pipe, as in US2020/0155016A. Moreover, since the frame 44 is supported by the delivery table 2, there no risk of forgetting the bag 42 nor the frame 44 nor the electronic control unit 46 when the patient on the delivery table must be moved from one room to another, in particular in case of an emergency. The monitoring system of the invention remains operational also in these circumstances.

The embodiment and variants mentioned here above may be combined to generate new embodiments of the invention, in the framework of the appended claims.

1. A monitoring system (4) for measuring a quantity of blood lost by a woman having given birth vaginally, this monitoring system including a port-partum blood flow collection container (42), this monitoring system including:
a weighing device (44, 446) configured to automatically and continuously weigh blood present in the container;
a computing unit (82) configured to automatically compute a blood volume (Vb) in the container (42), on the basis of an output signal (S446) of the weighing device;
a screen (48) configured to display a first value of the blood volume (Vb) in the container (42), computed by the computing unit (82);
wherein
the computing unit (82) is also configured to automatically compute a blood flow rate (Fb) of blood flowing into the container, on the basis of the output signal (S446) of the weighing device;
the screen (48) is configured to display a second value of the blood flow rate (Fb), computed by the computing unit (82).

2. The monitoring system of claim 1, wherein the weighing device includes a frame (44) for supporting the container and a weight sensor (446) mounted between two parts (442, 444) of the frame.

3. The monitoring system of claim 2, wherein the frame (44) includes a first part (442) provided with immobilization means (442C) for immobilizing the first part on a delivery table (2) and a second part (444) provided with holding means (444B) for holding the container (42) and wherein the weight sensor (446) includes at least one strain gauge (446A-4446D) connecting the first part (442) and the second part (444) of the frame.

4. The monitoring system of claim 2, wherein the container is a flexible bag (42) and the holding means include at least one clip (444B) configured to clip an edge (422) of the flexible bag in a configuration where the flexible bag hangs down from the frame (44), without touching on the ground (G).

5. The monitoring system of claim 2, wherein the computing unit (82) belongs to an electronic unit (46) configured to handle an output signal (S446) of the weight sensor (446) and to provide the screen (48) with a signal (S4) representative of the instantaneous blood volume (Vb) in the container (42) and of the instantaneous blood flow rate (Fb) toward the container.

6. The monitoring system of claim 5, wherein the electronic unit (46) includes an amplification module (84) for amplifying the output signal (S446) of the weight sensor (446) and a communication module (86) for sending to the screen (48) a signal (S4) including a data set with the blood volume (Vb) and the blood flow rate (Fb) computed by the computing unit (82).

7. The monitoring system of claim 5, wherein the electronic unit (46) is housed in a waterproof casing (462), cleanable according to hospital standards.

8. The monitoring system of claim 1, wherein the weighing device is incorporated in a scale, which supports the container.

9. A monitoring method for measuring a quantity of blood lost by a woman having given birth vaginally, this method including collection of post-partum blood flow in a container (42), wherein this method includes at least the following steps:
a) automatically and continuously weighing (1002) a quantity of blood present in the container (42); and
b) automatically computing (1004A) a volume (Vb) of the blood present in the container, based on a weight (W) determined at step a); c) automatically computing (1004B) a flow rate (Fb) of the blood flowing into the container, on the basis of variations of the volume computed at step b); and
d) displaying (1008) both
o a first value of the volume (Vb) computed at step b), o a second value of the blood flow rate (Fb) computed at step c).

10. The method of claim 9, wherein the blood volume (Vb) is computed at step b) on the basis of a measure of the weight of the container (42).

11. The method of claim 9, wherein the method includes at least the following steps consisting in:
e) comparing (1004C, 1004D) a value of the blood volume (Vb) to a first threshold value (V1) and/or a value of the blood flow rate (Fb) to a second threshold value (F1);
f) adapting (1008A, 1008B) step d) according to the result of the comparison of step e).

12. The method of claim 9, wherein an alarm is displayed (1008A, 1008B) at step d) if the result of the comparison of step e) shows that the blood volume value (Vb) is above the first threshold value (V1) and/or the blood flow rate (Fb) value is above the second threshold value (F1).

13. The method of claim 9, wherein the method includes a step of smoothing (1002A) over time instantaneous values of the weight (W) of the container (42), of the blood volume (Vb) and/or of the blood flow rate (Fb).

14. The method of claim 9, wherein it includes at least the following steps consisting in:
g) taking into account (1014) the fact that an object has been thrown in the container
h) subtracting the weight of the object thrown in the container from the weight (W) determined at step a).

15. The method of claim 9, wherein step d) occurs on a screen (48) and wherein the method includes a step, prior to step d), consisting in:
i) transmitting (1006) to the screen a signal (S4) including data to be displayed and relating to the blood volume (Vb) computed at step b) and to the blood flow rate (Fb) computed at step c);
and wherein the signal (S4) is preferably transmitted wirelessly at step i).

16. The method of claim 9, wherein step d) includes showing a curve (Cvb) representative of the blood volume (Vb) computed at step b) and/or a curve (CFb) representative of the blood flow rate (Fb) computed at step c).
